(19) United States
(12) Patent Application Publication
(10) Pub. No.: US 2026/0315754 A1
(43) Pub. Date: Oct. 15, 2026

(54) BALLOON FOR BALLOON CATHETER AND BALLOON CATHETER

(71) Applicant: KANEKA CORPORATION, Osaka (JP)

(72) Inventors: Masahiro Kojima, Osaka (JP); Takahisa Hamabuchi, Osaka (JP); Masato Tsueda, Osaka (JP)

(73) Assignee: KANEKA CORPORATION, Osaka (JP)

(21) Appl. No.: 19/274,079

(22) Filed: Jul. 18, 2025

Related U.S. Application Data
(63) Continuation of application No. PCT/JP2024/012664, filed on Mar. 28, 2024.

(30) Foreign Application Priority Data
Apr. 13, 2023 (JP) 2023-065587

(51) Int. Cl. A61M 25/10

(52) U.S. Cl. CPC A61M 25/1002; A61M 2025/1086

(57) ABSTRACT

A balloon for a balloon catheter including a straight portion having a ridge is provided. The ridge extends in a longitudinal direction and a plurality of notches arranged in the longitudinal direction are formed in the ridge. When the ridge is equally divided in the longitudinal direction into three sections that are a proximal section, an intermediate section, and a distal section, at least one of the notches is provided in each of the distal section and the intermediate section. A deepest depth of the at least one of the notches in the distal section is deeper than a deepest depth of the at least one of the notches in the intermediate section.

### TECHNICAL FIELD

One or more embodiments of the present invention relate to a balloon for a balloon catheter and a balloon catheter including the balloon.

### BACKGROUND

It is known that various diseases occur when a blood vessel, which is a flow path through which blood circulates in the body, becomes narrowed and the blood circulation stagnates. In particular, when stenosis occurs in a coronary artery that supplies blood to the heart, it may cause serious diseases such as angina pectoris and myocardial infarction. As one of the methods for treating such a stenosed site of a blood vessel, there is angioplasty (PTA, PTCA, etc.) that expands a stenosed site by using a balloon catheter.

Balloon catheters are known in each of which ridges are provided on a surface of a balloon (for example, PTLs 1 to 3). If such a balloon catheter is used, when a balloon is inflated, ridges of the balloon can bite into a stenosed site, and the stenosed site can be effectively expanded. On the other hand, a balloon provided with ridges tends to have high rigidity at its portions where the ridges are provided, and its flexibility in the longitudinal direction thereof is likely to decrease. In contrast, a balloon catheter in which notches are formed in ridges on a balloon surface is known (for example, PTLs 4 and 5). If such a balloon catheter is used, it is possible to maintain the flexibility of a balloon in the longitudinal direction of the balloon even if the balloon is provided with ridges.

### CITATION LIST

### Patent Literature

PTL 1: International Publication No. 2020/250611
PTL 2: Japanese Unexamined Patent Application Publication No. 2009-112361
PTL 3: Japanese Unexamined Patent Application Publication No. 2013-176507
PTL 4: International Publication No. 2012/099950
PTL 5: International Publication No. 2020/255923

A balloon with ridges provided on its surface and notches formed in the ridges can exhibit a scoring function by the ridges, while ensuring the flexibility of the balloon in the longitudinal direction of the balloon by the notches formed in the ridges. Since blood vessels have significantly curved portions, it is desirable that a balloon provided with ridges can be smoothly inserted even through such curved portions.

### SUMMARY

One or more embodiments of the present invention have been made in view of the above, and a balloon for a balloon catheter, the balloon having ridges on its surface and being capable of improving insertability into a curved portion of a blood vessel or the like, and a balloon catheter including the balloon, are provided.

A balloon for a balloon catheter and a balloon catheter including the balloon according to one or more embodiments of the present invention are as follows.
[1] A balloon for a balloon catheter having a longitudinal direction extending from a proximal side to a distal side, a radial direction orthogonal to the longitudinal direction, and a circumferential direction.

The balloon includes a straight portion, a proximal tapered portion located further toward a proximal side than the straight portion, and a distal tapered portion located further toward a distal side than the straight portion. The straight portion includes a cylindrical balloon main body and a ridge provided on an outer surface of the balloon main body, the ridge protruding outward in a radial direction and extending in a longitudinal direction.

A plurality of notches arranged in a longitudinal direction are formed in the ridge.

When the ridge is equally divided in a longitudinal direction into three sections that are a proximal section, an intermediate section, and a distal section, at least the distal section and the intermediate section are provided with the notches.

At least one of the notches that is provided in the distal section has a deepest depth greater than a deepest depth of at least one of the notches that is provided in the intermediate section.
[2] The balloon according to [1], wherein the notches are provided in each of the proximal section, the intermediate section, and the distal section, and a deepest depth of the notches provided in the distal section and a deepest depth of the notches provided in the proximal section are deeper than a deepest depth of the notches provided in the intermediate section.
[3] The balloon according to [1] or [2], wherein the number of the notches provided in the distal section is larger than the number of the notches provided in the intermediate section.
[4] The balloon according to any one of [1] to [3], wherein the number of the notches provided in the distal section and the number of the notches provided in the proximal section are larger than the number of the notches provided in the intermediate section.
[5] The balloon according to any one of [1] to [4], wherein the distal section is provided with a plurality of the notches having different depths.
[6] The balloon according to any one of [1] to [5], wherein a plurality of the notches having different depths are provided in each of the proximal section and the distal section.
[7] The balloon according to any one of [1] to [6], wherein the ridges are made of a resin, a metal, or a combination thereof.
[8] The balloon according to any one of [1] to [7], wherein a length of the straight tube portion in a longitudinal axis direction is 50 mm or more.
[9] A balloon catheter including the balloon according to any one of [1] to [8].

In the balloon for a balloon catheter of one or more embodiments of the present invention, a ridge is provided on an outer surface of a straight portion of the balloon, and thus, when a balloon catheter including the balloon is used, and the balloon is inflated at a stenosed site or the like of a blood vessel, the ridge can bite into the stenosed site or the like, and the stenosed site can be effectively expanded. In addition, notches are formed in the ridge of the balloon, and one of the notches that is deeper is formed in the distal section of the ridge. Thus, the flexibility of a distal portion of the balloon can be increased. Therefore, it becomes easier to insert the balloon into a curved portion.

### BRIEF DESCRIPTION OF DRAWINGS

FIG. 1 shows a configuration example of a balloon catheter according to one or more embodiments of the present invention and shows a side view of the balloon catheter.

FIG. 2 shows a cross-sectional view of the balloon catheter taken along line II-II of FIG. 1.

FIG. 3 shows a cross-sectional view of the balloon catheter taken along line III-III of FIG. 1.

FIG. 4 shows an example of a perspective view of a balloon included in the balloon catheter.

FIG. 5 shows a cross-sectional view of a straight portion of the balloon shown in FIG. 4, perpendicular to a longitudinal direction.

FIG. 6 shows a cross-sectional view of a ridge of the balloon shown in FIG. 5, perpendicular to the longitudinal direction.

FIG. 7 shows an example of a longitudinal cross-sectional view of the ridge of the balloon and a notch.

FIG. 8 shows another example of a longitudinal cross-sectional view of the ridge of the balloon and the notch.

FIG. 9 shows another example of a longitudinal cross-sectional view of the ridge of the balloon and the notch.

FIG. 10 shows another example of a longitudinal cross-sectional view of the ridge of the balloon and the notch.

FIG. 11 shows another example of a perspective view of the balloon included in a balloon catheter.

FIG. 12 shows another example of a cross-sectional view of the ridge of the balloon, taken perpendicular to the longitudinal direction.

### DETAILED DESCRIPTION

Hereinafter, one or more embodiments of the present invention will be described in detail based on an embodiment below. However, needless to say, the present invention is not limited to the embodiment below and may be appropriately modified within the scope of one or more embodiments of the present invention described above and below, and all of the modifications are included in the technical scope of the present invention. In each drawing, hatching, the reference signs of components, and the like may sometimes be omitted for convenience. In such cases, reference may be made to the specification or other drawings. Additionally, the dimensions of various components in the drawings may differ from the actual dimensions as priority is given to facilitating understanding of the features of one or more embodiments of the present invention.

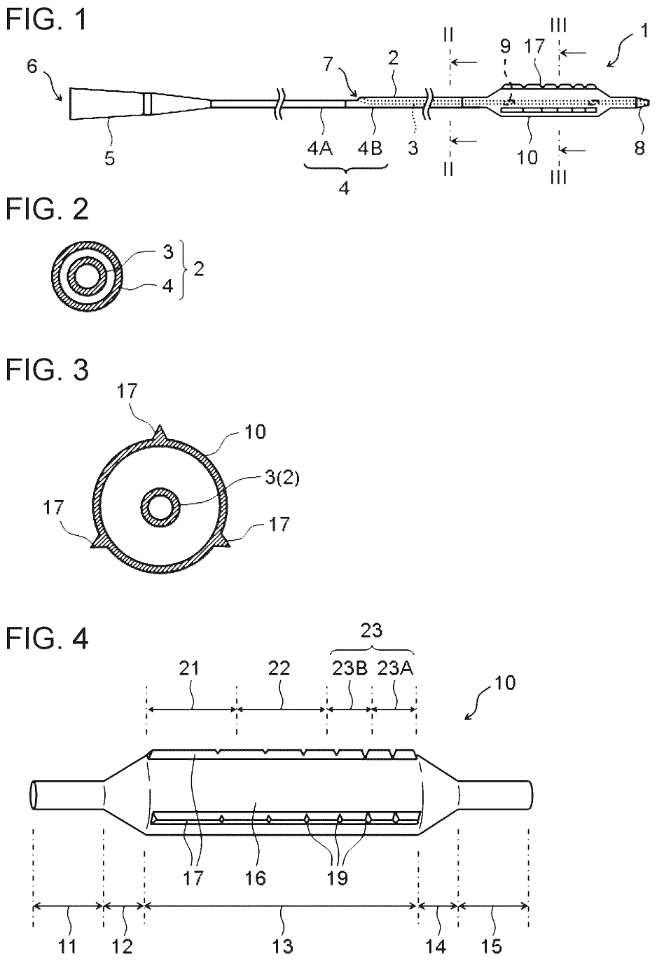

A configuration example of a balloon for a balloon catheter according to one or more embodiments of the present invention and a configuration example of a balloon catheter according to one or more embodiments of the present invention that includes the balloon will be described with reference to the drawings. FIGS. 1 to 4 show configuration examples of the balloon catheter. FIG. 1 shows a side view of the balloon catheter. FIG. 2 shows a cross-sectional view of the balloon catheter taken along line II-II of FIG. 1. FIG. 3 shows a cross-sectional view of the balloon catheter taken along line III-III of FIG. 1. FIG. 4 shows an example of a perspective view of the balloon included in the balloon catheter. FIG. 1 shows an example of the configuration of a rapid-exchange balloon catheter.

A balloon catheter 1 includes a shaft 2 and a balloon 10 provided outside the shaft 2. The balloon catheter 1 has a proximal side and a distal side, and the balloon 10 is provided on a distal portion of the shaft 2. The proximal side of the balloon catheter 1 refers to a direction toward a user's (operator's) hand with respect to a direction in which the balloon catheter 1 extends, and the distal side of the balloon catheter 1 refers to a direction opposite to the proximal side, that is, the direction toward a treatment target. The direction from the proximal side to the distal side of the balloon catheter 1 will be referred to as the longitudinal direction.

The balloon catheter 1 is configured such that a fluid is supplied to the inside of the balloon 10 through the shaft 2, and inflation and deflation of the balloon 10 can be controlled by using an indeflator (a pressure regulator for a balloon). The fluid may be a pressurized fluid that is pressurized by a pump or the like. Hereinafter, the fluid to be supplied to the inside of the balloon 10 will be referred to as a “balloon inflation fluid”.

The shaft 2 includes, for example, an inner shaft 3 and an outer shaft 4. The inner shaft 3 is disposed in the lumen of the outer shaft 4. The inner shaft 3 can function as an insertion path for a guide wire along which the shaft 2 is advanced, and when the balloon catheter 1 is used, the guide wire is inserted into the lumen of the inner shaft 3. The space between the inner shaft 3 and the outer shaft 4 can function as a flow path of the balloon inflation fluid.

In the rapid-exchange balloon catheter 1, a guidewire port 7 is provided at an intermediate position between the distal side and the proximal side of the shaft 2. The proximal end of the inner shaft 3 is connected to the guidewire port 7, and the distal end of the inner shaft 3 extends to the distal portion of the shaft 2, so that the insertion path for the guide wire is formed in such a manner as to extend from the guidewire port 7 to the distal portion of the shaft 2.

The outer shaft 4 may include a proximal outer shaft 4A and a distal outer shaft 4B, and in this case, the inner shaft 3 may be disposed in the lumen of the distal outer shaft 4B. The proximal outer shaft 4A and the distal outer shaft 4B may be made of the same material or may be made of different materials. For example, it may be preferable that the proximal outer shaft 4A be made of a resin or a metal, and that the distal outer shaft 4B be made of a resin. The outer shaft 4 is not necessarily divided into the proximal outer shaft 4A and the distal outer shaft 4B and may be formed of a single member. Alternatively, the proximal outer shaft 4A and the distal outer shaft 4B may each be further formed of a plurality of tube members.

A hub 5 may be provided on the proximal side of the shaft 2. The hub 5 may include a fluid injection portion 6 in communication with the flow path of the balloon inflation fluid in the shaft 2. The balloon 10, the shaft 2 (the inner shaft 3 and the outer shaft 4), and the hub 5 can be joined to each other by using joining means that is known in the related art, such as an adhesive or thermal welding.

Although not shown in the drawings, the balloon catheter may be an over-the-wire balloon catheter in which an inner shaft extends from a distal portion of a shaft to a proximal portion of the shaft and in which an insertion path for a guide wire is formed in such a manner as to extend from the distal side of the shaft to the proximal side of the shaft. In this case, it may be preferable that the flow path of the balloon inflation fluid and the insertion path for the guide wire, which are provided in the shaft, extend to the hub, and that the hub be configured to include the fluid injection portion communicating with the flow path of the balloon inflation fluid and a treatment portion communicating with the insertion path for the guide wire. It may be preferable that the hub have a bifurcated structure in which the fluid injection portion is provided in one of bifurcated portions and in which the treatment portion is provided in the other bifurcated portion.

The outer surface of the shaft 2 may be coated. In the rapid-exchange balloon catheter 1, one or both of the outer surface of the proximal outer shaft 4A and the outer surface of the distal outer shaft 4B may be coated, and the outer surfaces of both the proximal outer shaft 4A and the distal outer shaft 4B may be coated. In the over-the-wire balloon catheter, the outer surface of the outer shaft may be coated as appropriate.

The coating can be a hydrophilic coating or a hydrophobic coating, depending on the purpose. The outer surface of the shaft 2 can be coated by immersing the shaft 2 in a hydrophilic coating agent or a hydrophobic coating agent, by applying a hydrophilic coating agent or a hydrophobic coating agent to the outer surface of the shaft 2, or by coating the outer surface of the shaft 2 with a hydrophilic coating agent or a hydrophobic coating agent. The coating agent may contain a drug or an additive.

Examples of the hydrophilic coating agent include hydrophilic polymers such as polyvinyl alcohol, polyethylene glycol, polyacrylamide, polyvinylpyrrolidone, and methyl vinyl ether-maleic anhydride copolymer, and hydrophilic coating agents and the like made of any combination of these.

Examples of the hydrophobic coating agents include polytetrafluoroethylene (PTFE), fluorinated ethylene propylene (FEP), perfluoroalkoxyalkane (PFA), silicone oil, hydrophobic urethane resin, carbon coating, diamond coating, diamond-like carbon (DLC) coating, ceramic coating, and substances and the like with low surface free energy terminated with alkyl groups or perfluoroalkyl groups.

At a distal end portion of the balloon catheter 1, a distal tip 8 may be provided. The distal tip 8 may be provided, as a separate component from the inner shaft 3, at a position further toward the distal side than the distal end of the inner shaft 3, or the inner shaft 3 may extend to a position further toward the distal side than the distal end of the balloon 10 such that a distal end portion of the inner shaft 3 functions as the distal tip 8.

In order to enable confirmation of the position of the balloon 10 under X-ray fluoroscopy, one or more radiopaque markers 9 may be provided at a portion of the shaft 2 where the balloon 10 is located in the longitudinal direction. The one or more radiopaque markers 9 can be placed, for example, on the inner shaft 3 located inside the balloon 10, may be positioned at locations corresponding to both ends of a straight portion of the balloon 10, or may be positioned at a location corresponding to the center of the straight portion of the balloon 10.

The balloon 10 has a longitudinal direction and a radial direction, and is formed in a tubular shape having openings on the proximal side and the distal side (see FIG. 4). The radial direction of the balloon 10 is a direction orthogonal to the longitudinal direction and refers to a direction extending radially from the center of the balloon 10. The balloon 10 also has a circumferential direction as a direction along the outer circumference of the balloon 10 in an inflated state in a cross section of the balloon 10 perpendicular to the longitudinal direction.

The balloon 10 includes a straight portion 13, a proximal tapered portion 12 located further toward the proximal side than the straight portion 13, and a distal tapered portion 14 located further toward the distal side than the straight portion 13 with respect to the longitudinal direction. The straight portion 13 is formed in a substantially cylindrical shape extending in the longitudinal direction, and is formed to have the largest length in the radial direction (outer diameter) in the balloon 10. The proximal tapered portion 12 is located on the proximal side of the straight portion 13 and connected to the proximal end of the straight portion 13. The proximal tapered portion 12 is formed such that the outer diameter thereof decreases with increasing distance from the straight portion 13. The distal tapered portion 14 is located on the distal side of the straight portion 13 and connected to the distal end of the straight portion 13. The distal tapered portion 14 is formed such that the outer diameter thereof decreases with increasing distance from the straight portion 13. The balloon 10 may further include a proximal sleeve portion 11 located further toward the proximal side than the proximal tapered portion 12 and a distal sleeve portion 15 located further toward the distal side than the distal tapered portion 14. The proximal sleeve portion 11 is located on the proximal side of the proximal tapered portion 12 and is connected to the proximal end of the proximal tapered portion 12. The proximal sleeve portion 11 is formed in a substantially cylindrical shape. The distal sleeve portion 15 is located on the distal side of the distal tapered portion 14 and is connected to the distal end of the distal tapered portion 14. The distal sleeve portion 15 is formed in a substantially cylindrical shape.

By configuring the balloon 10 as described above, when the balloon 10 is inflated at a stenosed site, the straight portion 13 comes into sufficient contact with the stenosed site, making it easier to perform treatment such as expansion of the stenosed site. In addition, since the balloon 10 includes the proximal tapered portion 12 and the distal tapered portion 14, when the balloon 10 is deflated, the outer diameter of a proximal end portion of the balloon 10 and the outer diameter of a distal end portion of the balloon 10 can be reduced so as to reduce the difference in diameter between the shaft 2 and the balloon 10, so that it becomes easier to insert the balloon 10 into a body cavity, a forceps channel of an endoscope, or a delivery catheter such as a guiding catheter.

In the distal portion of the shaft 2, it may be preferable that the inner shaft 3 extend to a position further toward the distal side than the distal end of the outer shaft 4, and that the inner shaft 3 extend from the proximal sleeve portion 11 to the distal sleeve portion 15 through the internal space of the balloon 10. It may be preferable that the outer surface of the inner shaft 3 be joined to the inner surface of the distal sleeve portion 15 of the balloon 10, and that the outer surface of the outer shaft 4 be joined to the inner surface of the proximal sleeve portion 11 of the balloon 10. With the distal portion of the shaft 2 configured as described above, the balloon inflation fluid can be supplied to the internal space of the balloon 10 through the space between the inner shaft 3 and the outer shaft 4.

The size of the balloon 10 is not particularly limited. The size of the balloon 10 can be appropriately set, for example, with the length of the straight portion 13 in the longitudinal direction ranging from 4 mm to 400 mm, and the outer diameter of the straight portion 13 ranging from 1 mm to 30 mm.

The balloon 10 (particularly a balloon main body 16) may be made of a resin. The resin may be a thermoplastic resin. This makes it easier to manufacture the balloon 10 by molding. Examples of the resin out of which the balloon 10 is made include polyolefin resins such as polyethylene, polypropylene, and ethylene-propylene copolymer; polyester resins such as polyethylene terephthalate and polyester elastomers; polyurethane resins such as polyurethane and polyurethane elastomers; polyphenylene sulfide resin; polyamide resins such as polyamide and polyamide elastomers; fluorine-based resins; silicone resins; and natural rubbers such as latex rubber. Only one of these may be used, or two or more of these may be used in combination. Among these, polyamide resins, polyester resins, and polyurethane resins may be used. In particular, it may be preferable to use an elastomer resin from the viewpoint of thinning and flexibility of the balloon 10. Examples of materials suitable for the balloon 10 among polyamide resins include nylon 12, nylon 11, and the like, and nylon 12 may be used because it can be relatively easily molded at the time of blow molding. In addition, from the viewpoint of thinning and flexibility of the balloon 10, polyamide elastomers such as polyether ester amide elastomer and polyamide ether elastomer may be used. Among these, polyether ester amide elastomer may be used from the viewpoint of high yield strength and favorable dimensional stability of the balloon 10.

The balloon 10 includes ridges 17 formed on the outer surface of the straight portion 13. Providing the ridges 17 on the outer surface of the straight portion 13 gives the balloon 10 a scoring function, and when the balloon 10 is inflated at a stenosed site of a blood vessel, the ridges 17 can bite into the stenosed site that has been calcified, thereby forming cracks in the stenosed site. Therefore, it is possible to expand the stenosed site while suppressing dissection of the blood vessel. Additionally, it is possible to increase the pressure resistance of the balloon 10 and suppress over-inflation of the balloon 10 during pressurization of the balloon 10. Note that, although the balloon 10 can also be used for treatment of a stenosed site or a lesion in a body cavity other than a blood vessel, the following description will mainly focus on the case where the balloon 10 is applied to vascular treatment.

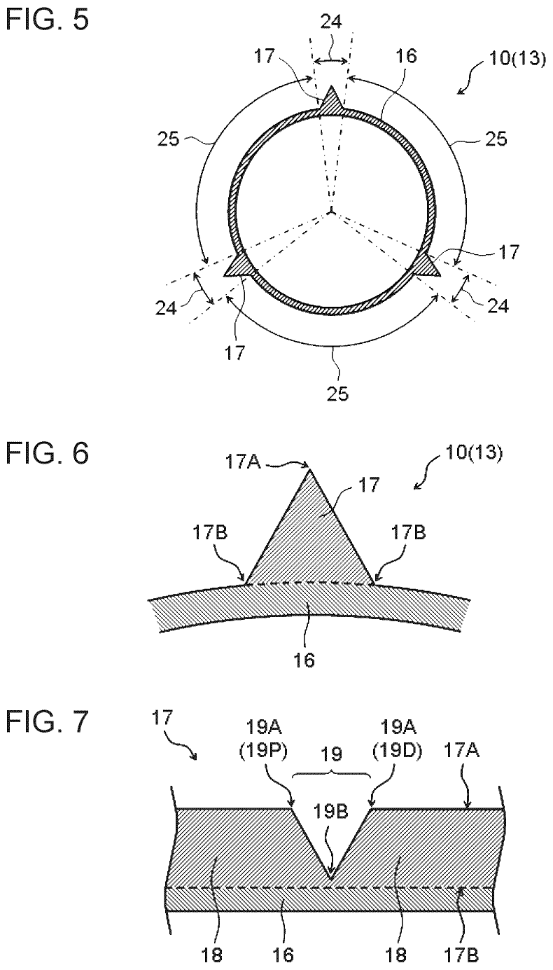

The ridges 17 of the balloon 10 will be described in detail with reference to FIGS. 5 and 6. FIG. 5 shows a cross-sectional view of the straight portion 13 of the balloon 10, perpendicular to the longitudinal direction, and FIG. 6 shows an enlarged cross-sectional view of one of the ridges 17 of the balloon 10. FIG. 5 shows a configuration example of the straight portion 13 of the balloon 10, which is shown in FIG. 4, in a cross section perpendicular to the longitudinal direction, and the ridges 17 are provided at three positions in the circumferential direction of the straight portion 13.

The straight portion 13 of the balloon 10 includes a cylindrical balloon main body 16, and the ridges 17 are provided on the outer surface of the balloon main body 16. The ridges 17 are provided so as to protrude in the radial direction outward from the outer surface of the balloon main body 16. In the balloon 10, a ridge-present region 24 and a ridge-absent region 25 are formed on the outer surface of the straight portion 13 by providing the ridges 17. As will be described later, the ridge-present region 24 includes portions of the ridges 17 in each of which a notch 19 is formed. The outer surface of the straight portion 13 may be formed to be flat in the ridge-absent region 25. For example, the outer surface of the straight portion 13 may not be formed to be recessed in a portion of the ridge-absent region 25. This facilitates uniform inflation of the balloon 10 and makes it easier for the ridges 17 to exhibit a desired scoring function. The phrase “the outer surface of the straight portion 13 is formed to be flat in the ridge-absent region 25” refers to the case where the ridge-absent region 25 has an arched shape obtained by bending a flat surface and where no irregularities are formed on the flat surface bent in an arch shape. The irregularities do not include surface roughness that is inevitably formed in manufacturing.

Each of the ridges 17 includes a top portion 17A and a base portion 17B. In each of the ridges 17, the top portion 17A refers to the tip of the ridge 17, that is, the outermost portion of the ridge 17 in the radial direction, and the base portion 17B refers to a boundary between the ridge 17 and the balloon main body 16, that is, the innermost portion of the ridge 17 in the radial direction.

The ridges 17 can be made of, for example, a resin. If the ridges 17 are made of a resin, the balloon 10 including the ridges 17 can be manufactured by resin molding, which facilitates manufacturing. In this case, the ridges 17 and the balloon main body 16 may be made of the same resin, and the ridges 17 and the balloon main body 16 may be integrally molded. The balloon main body 16 may have an inner layer and an outer layer, and in this case, the ridges 17 may be made of the same resin as that of the outer layer of the balloon main body 16. As a result, the ridges 17 are less likely to unintentionally fall off from the balloon main body 16. Alternatively, if the resin constituting the ridges 17 and the resin constituting the balloon main body 16 are compatible with each other to some extent, the ridges 17 and the balloon main body 16 may be made of different resins.

The ridges 17 may be made of a metal or may be made of a combination of a metal and a resin. In this case, it may be preferable that portions of the ridges 17 including the top portions 17A be made of a metal. This makes it easier for the ridges 17 to form cracks in a stenosed site or to cut open the stenosed site when the balloon 10 is inflated. For example, the entire ridges 17 may be made of a metal, or portions of the ridges 17 including the base portions 17B may be made of a resin while portions of the ridges 17 including the top portions 17A may be made of a metal. Therefore, the ridges 17 may be made of a resin, a metal, or a combination of these.

In the straight portion 13, the balloon main body 16 is defined as a portion having a tubular shape. A portion of the straight portion 13 excluding the ridges 17 protruding outward in the radial direction corresponds to the balloon main body 16. The balloon main body 16 can be regarded as having a cylindrical outer surface. Therefore, in a cross section of the straight portion 13 perpendicular to the longitudinal direction, the outer shape of the balloon main body 16 is formed in a substantially circular shape, so that the balloon main body 16 and the ridges 17 can be distinguished from each other. The ridge-present region 24 is constituted by the balloon main body 16 and the ridges 17, and the ridge-absent region 25 is constituted by the balloon main body 16.

The ridges 17 are provided on the outer surface of the straight portion 13 in such a manner as to extend in a ridge-like manner. The ridges 17 extend substantially parallel to the longitudinal direction of the balloon 10. The plurality of ridges 17 are provided at different positions on the straight portion 13 of the balloon 10 in the circumferential direction. That is, the ridges 17 are provided at a plurality of positions in the circumferential direction of the balloon 10. In this case, the ridges 17 may be arranged at substantially equal intervals in the circumferential direction of the straight portion 13 of the balloon 10. As a result, when the balloon 10 is inflated, it is possible to form cracks in a plurality of portions of the stenosed site. In the circumferential direction of the balloon 10, the ridges 17 may be provided at two or more positions, or at three or more positions, and at twelve or less positions, at ten or less positions, or at eight or less positions. In this case, it may be preferable that the interval between the ridges 17 in the circumferential direction be longer than the length of one of the ridges 17 in the circumferential direction.

The plurality of ridges 17 may be provided at substantially the same position in the longitudinal direction. That is, the proximal ends of the plurality of ridges 17 may be located at substantially the same position in the longitudinal direction, and the distal ends of the plurality of ridges 17 may be located at substantially the same position in the longitudinal direction.

The cross-sectional shape of each of the ridges 17 is not particularly limited. For example, the shape of each of the ridges 17 in a cross section of the straight portion 13 perpendicular to the longitudinal direction may be a polygonal shape, such as a triangular shape or a rectangular shape, a partial shape of a circle, such as a semicircular shape or a sector shape, a wedge shape, a convex shape, a spindle shape, an irregular shape, or the like. The polygonal shape includes a rounded polygonal shape with rounded corners and a polygonal shape with at least partially curved sides, in addition to a polygonal shape with clearly defined corner vertices and the straight sides. Note that each of the ridges 17 may be formed such that the width thereof gradually decreases toward the top portion 17A.

In a cross section of the straight portion 13 perpendicular to the longitudinal direction, the height of each of the ridges 17 may be 0.2 times or more the width (maximum width) of the ridge 17. If the ridges 17 are formed in this manner, when the balloon 10 is inflated at a stenosed site, the ridges 17 easily bite into the stenosed site, and the scoring function provided by the ridges 17 can be enhanced. The width of each of the ridges 17 described here refers to the length of the ridge 17 in the circumferential direction. Each of the ridges 17 may be formed so as to be widest at the base portion 17B, so that the ridge 17 is stably provided on the outer surface of the balloon main body 16. The height of each of the ridges 17 may be 0.4 times or more the width of the ridge 17, or 0.7 times or more the width of the ridge 17. On the other hand, the height of each of the ridges 17 may be 2.0 times or less the width of the ridges 17, 1.8 times or less the width of the ridges 17, or 1.5 times or less the width of the ridges 17. This makes it easier to ensure the flexibility of the balloon 10 at the portions of the balloon 10 where the ridges 17 are present.

In the straight portion 13, the thickness of the portion where the ridges 17 are provided, that is, the thickness of the ridge-present region 24, may be formed to be thicker than the thickness of the portion where the ridges 17 are not provided, that is, the thickness of the ridge-absent region 25. This can enhance the scoring function provided by the ridges 17. The thickness (maximum thickness) of the ridge-present region 24 may be 1.5 times or more, 2.0 times or more, or 2.5 times or more the thickness (maximum thickness) of the ridge-absent region 25. The upper limit of the thickness of the ridge-present region 24 is not particularly limited and may be, for example, 30 times or less, 20 times or less, or 10 times or less the thickness of the ridge-absent region 25.

In the balloon 10, the ridges 17 may be provided in a range of 60% or more, in a range of 70% or more, or in a range of 80% or more of the straight portion 13 in the longitudinal direction of the straight portion 13. This enables formation of cracks in a wide area of a stenosed site when the balloon 10 is inflated. The ridges 17 may be provided in a range of 90% or more of the straight portion 13 in the longitudinal direction, may be provided over substantially the entire straight portion 13 in the longitudinal direction, or may be provided on the outer surface of the proximal tapered portion 12 and/or the outer surface of the distal tapered portion 14.

The balloon 10 may include an inner ridge (not shown) provided on the inner surface of the balloon 10 in such a manner as to protrude toward the inner side in the radial direction. At least one of the ridges 17 and the inner ridge may be arranged at the same position in the longitudinal direction of the balloon 10 and the circumferential direction of the balloon 10, and those may be integrally formed, so that a portion of the balloon 10 may be formed to be thick.

The balloon 10 provided with the ridges 17 tends to have high rigidity at the portions there of where the ridges 17 are provided. Therefore, the balloon 10 provided with ridges 17 is more likely to have reduced flexibility in the longitudinal direction, compared with the balloon 10 without the ridges 17. For example, in a shunt that is formed during hemodialysis, a blood vessel bends significantly at an arteriovenous anastomosis site. When passing a balloon through such a location, there is a risk that the balloon with ridges may have difficulty passing through the arteriovenous anastomosis site. In the case of a lower limb balloon, the balloon is inserted into an iliac artery during treatment. However, at the bifurcation where the left and right iliac arteries branch from the abdominal aorta, a blood vessel bends significantly. Therefore, if the lower limb balloon is provided with ridges, there is a risk that it may be difficult to pass the balloon from one of the left and right iliac arteries to the other. In particular, since a lower limb balloon is long, there is a higher risk that the balloon cannot pass through a location where a blood vessel bends significantly. Therefore, as shown in FIG. 4, the notch 19 is formed in each of the ridges 17 of the balloon 10. By forming the notch 19 in each of the ridges 17, the flexibility of the balloon 10 in the longitudinal direction can be increased.

The notch 19 may be formed in each of the ridges 17. This makes it possible to increase the flexibility of the balloon 10 regardless of the direction in which the balloon 10 is bent.

The number of the notches 19 formed in each of the ridges 17 is not particularly limited as long as it is one or more. However, from the viewpoint of increasing the flexibility of the balloon 10, the number of the notches 19 formed in each of the ridges 17 may be two or more, or three or more. On the other hand, from the viewpoint of ensuring the scoring function of the balloon 10, the number of the notches 19 formed in each of the ridges 17 may be 20 or less, 16 or less, 12 or less, or 8 or less.

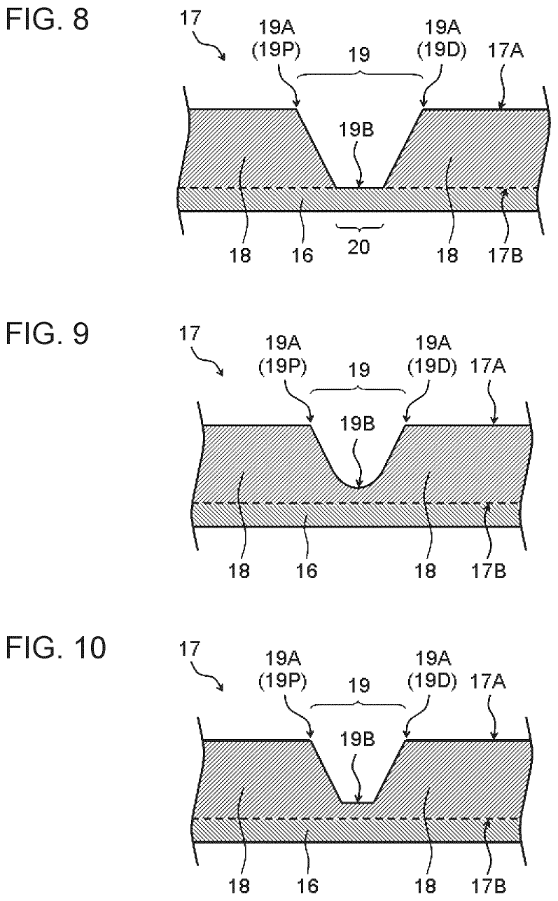

Each of the notches 19 may be formed in such a manner that it partially cuts off the top portion 17A of the corresponding ridge 17, which extends in the longitudinal direction. FIGS. 7 to 10 show various formation examples of one of the notches 19 and each show a cross-sectional view taken along the longitudinal direction passing through the top portion 17A of one of the ridges 17 (to be specific, a cross-sectional view taken along both the longitudinal direction and the radial direction passing through the top portion 17A of the ridge 17), illustrating the ridge 17 and the notch 19. As shown in FIG. 8, the notch 19 may be formed so as to extend from the top portion 17A to the base portion 17B of the ridge 17, and as shown in FIGS. 7, 9, and 10, the notch 19 may be formed so as to extend from the top portion 17A to an intermediate position between the top portion 17A and the base portion 17B of the ridge 17. In the former case, the depth of each of the notches 19 is equal to the height of the corresponding ridge 17. In the latter case, the depth of each of the notches 19 is formed to be shorter than the height of the corresponding ridge 17.

Each of the notches 19 has a bottom portion 19B and top portions 19A. The bottom portion 19B corresponds to the innermost portion of the notch 19 in the radial direction, and the top portions 19A corresponds to the outermost portions of the notch 19 in the radial direction. The top portions 19A of each of the notches 19 correspond to a proximal end 19P and a distal end 19D of the notch 19 and coincide with the top portion 17A of the corresponding ridge 17. The length in the radial direction from each of the top portions 19A to the bottom portion 19B of each of the notches 19 is the depth of the notch 19.

The ridges 17 are divided into a plurality of ridge segments 18 by the notches 19. As shown in FIG. 8, in the case where one of the notches 19 is formed so as to extend from the top portion 17A to the base portion 17B of one of the ridges 17, an interrupted portion 20 of the ridge 17 is formed by the notch 19, and the ridge segments 18 are arranged on the proximal side and the distal side of the interrupted portion 20. The interrupted portion 20 has a predetermined length in the longitudinal direction, and the ridge 17 is formed by alternately arranging the ridge Segments 18 and the interrupted portion 20 in the longitudinal direction. In the case where the notch 19 is formed to extend from the top portion 17A to an intermediate position between the top portion 17A and the base portion 17B of the ridge 17, the ridge 17 is divided into the ridge segment 18 on the proximal side and the ridge segment 18 on the distal side, with the bottom portion 19B of the notch 19 as the boundary.

The shape of each of the notches 19 is not particularly limited. For example, in a cross section taken along the longitudinal direction and passing through the top portion 17A of one of the ridges 17, each of the notches 19 may be formed in a V-shape as shown in FIG. 7, a U-shape as shown in FIG. 9, or in a shape obtained by removing one side of a rectangle as shown in FIGS. 8 and 10. It may be preferable that, in a cross section taken along the longitudinal direction and passing through the top portion 17A of one of the ridges 17, the outer edge of the notch 19 have a portion extending radially outward and linearly inclined proximally, and a portion extending radially outward and linearly inclined distally on the distal side of the former portion. The portion of the outer edge of the notch 19 that extends in an inclined manner toward the proximal side or the distal side may extend in a linearly inclined manner, may extend in a curved inclined manner, or may be a combination thereof. The entire outer edge of the notch 19 on the proximal side may be formed so as to extend from the bottom portion 19B of the notch 19 radially outward and linearly inclined proximally, and the entire outer edge of the notch 19 on the distal side may be formed so as to extend from the bottom portion 19B of the notch 19 radially outward and linearly inclined distally. Hereinafter, the length and shape of each of the notches 19 will be described in detail. However, these are defined based on the length and shape of the notch 19 in a cross section taken along the longitudinal direction and passing through the top portion 17A of the corresponding ridge 17. The wording “a cross section taken along the longitudinal direction and passing through the top portion 17A of the corresponding ridge 17” refers to a cross section taken along the longitudinal direction and the radial direction and passing through the top portion 17A of the ridge 17.

The length of each of the notches 19 in the longitudinal direction may be shorter than the length of each of the ridge segments 18 in the longitudinal direction. Specifically, in each of the ridges 17, the length of the notch 19 in the longitudinal direction (the length of each of the notches 19 in the longitudinal direction in the case where multiple notches 19 are provided in each ridge 17) may be shorter than the length of any one of the ridge segments 18 in the longitudinal direction. In each of the ridges 17, the length of the notch 19 in the longitudinal direction (the length of each of the notches 19 in the longitudinal direction in the case where multiple notches 19 are provided in each ridge 17) may be 0.5 times or less, 0.3 times or less, or 0.2 times or less the average value of the lengths of the ridge segments 18 in the longitudinal direction. This makes it easier to ensure the scoring function provided by the ridges 17. Note that the length of the notch 19 in the longitudinal direction refers to the distance between top portions of the ridge segments 18 located on the proximal and distal sides across the notch 19, that is, the distance between the distal end of the top portion of the ridge segment 18 located on the proximal side of the notch 19 and the proximal end of the top portion of the ridge segment 18 located on the distal side of the notch 19.

In each of the ridges 17, the total length of the notch 19 in the longitudinal direction may be 20% or less, 15% or less, or 10% or less of the length of the ridge 17 in the longitudinal direction. This makes it easier to ensure the scoring function provided by the ridges 17. The length of each of the ridges 17 in the longitudinal direction is determined as follows. Among the plurality of ridge segments 18 constituting each ridge 17, the proximal end of the ridge segment 18 on the most proximal side is the proximal end of the ridges 17, the distal end of the ridge segment 18 on the most distal side is the distal end of the ridges 17, and the length in the longitudinal direction from the proximal end to the distal end of the ridges 17 is the length in the longitudinal direction of the ridges 17.

In each of the notches 19, the length of the notch 19 in the longitudinal direction may be 0.2 times or more, 0.3 times or more, or 0.5 times or more the depth of the notch 19. This makes it easier to increase the flexibility of the straight portion 13 of the balloon 10 in the longitudinal direction. In each of the notches 19, the length of the notch 19 in the longitudinal direction may be 5 times or less, 3 times or less, or 2 times or less the depth of the notch 19. This makes it easier to ensure the scoring function of the balloon 10.

It may be preferable that the length of a portion of each of the notches 19 where the bottom portion 19B extends parallel to the longitudinal direction not be formed very long compared with the length of the notch 19 in the longitudinal direction (the distance between the top portions of the ridge segments 18 on the proximal and distal sides with the notch 19 interposed therebetween) (see FIGS. 8 and 10). In each of the notches 19, the length of the portion of the notch 19 where the bottom portion 19B extends parallel to the longitudinal direction may be 0.5 times or less, 0.3 times or less, or 0.2 times or less the length of the notch 19 in the longitudinal direction. This facilitates smooth bending of the balloon 10 at the notches 19 and makes it easier to ensure the scoring function by the ridges 17. The bottom portion 19B of each of the notches 19 does not necessarily include a portion extending parallel to the longitudinal direction. In the case where each of the notches 19 is formed in such a manner as to extend from the top portion 17A to the base portion 17B of the corresponding ridge 17, the length of the interrupted portion 20 of the ridge 17 in the longitudinal direction (the length of the interrupted portion 20 on the outer surface of the balloon main body 16 in the longitudinal direction) corresponds to the length of the bottom portion 19B of the notch 19 in the longitudinal direction.

In each of the ridges 17, as shown in FIG. 4, the notch 19 formed in the distal portion is deeper than the other notches 19. Specifically, when the ridge 17 is equally divided in the longitudinal direction into three sections, which are a proximal section 21, an intermediate section 22, and a distal section 23, at least the distal section 23 and the intermediate section 22 are each provided with the notches 19, and the deepest depth of the notches 19 provided in the distal section 23 is deeper than the deepest depth of the notches 19 provided in the intermediate section 22. By forming the notches 19 in the ridge 17 in this manner, it is possible to increase the flexibility of a distal portion of the balloon 10 (specifically, the portion of the balloon 10 corresponding to the distal sections 23 of the ridges 17 in the longitudinal direction).

When the balloon 10 is inserted into a curved portion of a blood vessel, the flexibility of the distal portion of the balloon 10 is particularly important. If the distal portion of the balloon 10 cannot be inserted into the curved portion of the blood vessel, a portion of the balloon 10 that is located further toward the proximal side than the distal portion also cannot be inserted into the curved portion, making it impossible to perform treatment using the balloon 10. In this case, if the balloon 10 is pushed in so as to forcibly pass the distal portion of the balloon 10 through the curved portion of the blood vessel, the possibility of damaging the blood vessel increases. In contrast, if the distal portion of the balloon 10 can once be passed through the curved portion, the balloon 10 can be inserted through the curved portion while suppressing the risk of blood vessel damage even if the portion on the proximal side of the distal portion of the balloon 10 is slightly inferior in flexibility.

The proximal section 21, the intermediate section 22, and the distal section 23 of each of the ridges 17 are defined as follows. In each of the ridges 17, when the length in the longitudinal direction from the proximal end to the distal end of the ridge 17 is L, the ridge 17 is divided into three sections, each having a length of L/3. The section on the most proximal side is defined as the proximal section 21, the section on the most distal side is defined as the distal section 23, and the section between the proximal section 21 and the distal section 23 is defined as the intermediate section 22. The positions of the notches 19 in the ridges 17 in the longitudinal direction, that is, whether each of the notches 19 is located in the proximal section 21, the intermediate section 22, or the distal section 23 of each of the ridges 17, is determined based on the position of the bottom portion 19B of the notch 19 in a cross section taken along the longitudinal direction and passing through the top portion 17A of the corresponding ridge 17. When the bottom portion 19B of the notch 19 is formed to have a predetermined length in the longitudinal direction, a midpoint of the bottom portion 19B of the notch 19 in the longitudinal direction is defined as the position of the notch 19 on the ridge 17 in the longitudinal direction. When the bottom portion 19B of the notch 19 is positioned exactly at the boundary between the proximal section 21 and the intermediate section 22 or at the boundary between the intermediate section 22 and the distal section 23, the notch 19 is determined to be present in both sections but not to belong either section.

A plurality of notches 19 having different depths may be provided in the distal section 23. A plurality of notches 19 having different depths may be provided in the intermediate section 22. In each of the ridges 17, the deepest notch 19 among the notches 19 provided in the distal section 23 may be formed deeper than the deepest notch 19 among the notches 19 provided in the intermediate section 22. In the balloon 10, in at least one of the ridges 17, the notches 19 may be formed such that the deepest depth of the notches 19 provided in the distal section 23 is deeper than the deepest depth of the notches 19 provided in the intermediate section 22. In each of the ridges 17, the notches 19 may be formed in this manner. Also in the following description of the notches 19, the notches 19 may be formed as in the description in at least one of the ridges 17, or the notches 19 may be formed as in the description in each of the ridges 17.

In the case where the notches 19 are provided in the ridges 17 as described above, the notches 19 is essentially provided in the distal sections 23 and the intermediate sections 22 of the ridges 17, and the notches 19 may or may not be provided in the proximal sections 21 of the ridges 17. When the balloon 10 passes through a curved portion by being withdrawn after treatment using the balloon 10, since the shaft 2 of the catheter 1 has already passed through the curved portion, the balloon 10 can pass through the curved portion more easily than when the balloon 10 is advanced and inserted into the curved portion. Therefore, even if the notches 19 are not provided in the proximal sections 21 of the ridges 17, the balloon 10 can be withdrawn and passed through the curved portion. However, it may be preferable that the notches 19 also be provided in the proximal sections 21 of the ridges 17 in order to improve insertability into such a curved portion when withdrawing the balloon 10.

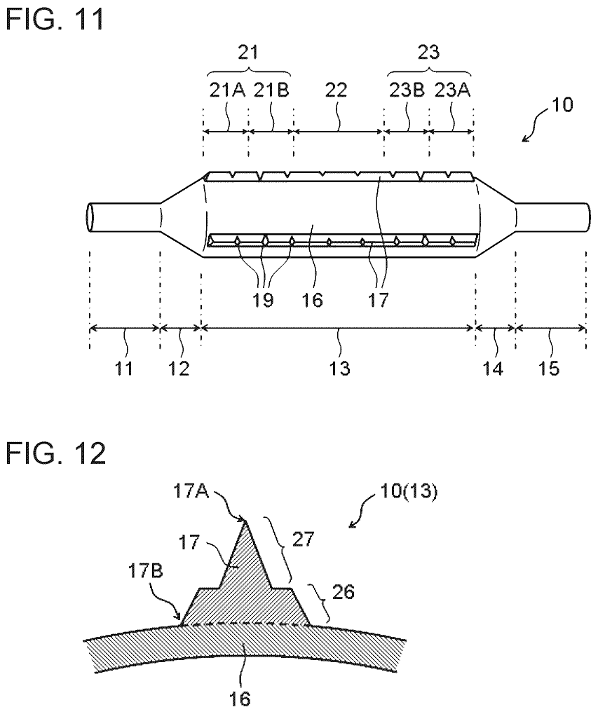

FIG. 11 shows another example of a perspective view of the balloon 10 included in the balloon catheter 1. As shown in FIG. 11, it may also be preferable that the notches 19 be provided in the respective sections such that the deepest depth of the notches 19 provided in the distal section 23 and the deepest depth of the notches 19 provided in the proximal section 21 are each deeper than the deepest depth of the notches 19 provided in the intermediate section 22. By providing the notches 19 in the ridge 17 in this manner, the balloon 10 can easily pass through a curved portion both when advancing the balloon 10 and when withdrawing the balloon 10.

In one embodiment, a plurality of notches 19 having different depths may be provided in the distal sections 23. By providing the notches 19 in this manner, the flexibility of the distal sections 23 of the ridges 17 can be enhanced, and a certain level of rigidity of the ridges 17 can be ensured at the notches 19 that are relatively shallow. Therefore, it becomes easier to advance and insert the balloon 10 into a curved portion, and the scoring function provided by the ridges 17 can be ensured in the distal sections 23 of the ridges 17.

The deepest depth of the notches 19 provided in each of the distal sections 23 may be 1.5 times or more, 1.8 times or more, or 2 times or more the deepest depth of the notch 19 provided in the corresponding intermediate section 22. As a result, the flexibility of the distal sections 23 of the ridges 17 can be increased, and when the balloon 10 is advanced and inserted into a curved portion, the followability of the distal portions of the balloon 10 can be increased. On the other hand, the deepest depth of the notches 19 provided in each of the distal sections 23 may be 10 times or less, 8 times or less, or 5 times or less the deepest depth of the notch 19 provided in the corresponding intermediate section 22. This makes it possible to increase the flexibility of the intermediate sections 22 of the ridges 17.

The distal sections 23 may be provided with the notches 19 as follows. That is, when each of the distal sections 23 is bisected in the longitudinal axis direction into a first distal section 23A and a second distal section 23B from the distal side, it may be preferable that at least one of the notches 19 be provided in at least the second distal section 23B. In the case where multiple notches 19 are provided in the distal section 23, it may be preferable that the notch 19 that has the deepest depth in the distal section 23 be provided in the second distal section 23B. By providing the notches 19 in this manner, even if the balloon 10 has a long length in the longitudinal direction, the overall flexibility of the balloon 10 can be more easily ensured. For example, the notch 19 that has the deepest depth in the distal section 23 may be provided in the second distal section 23B, and the notch 19 that has a shallower depth may be provided in the first distal section 23A. This ensures the overall flexibility of the balloon 10, making it easier to advance and insert the balloon 10 into a curved portion, while ensuring the scoring function provided by the ridges 17 in the distal sections 23 of the ridges 17.

In the case where the notches 19 are also provided in the proximal sections 21 of the ridges 17, the plurality of notches 19 having different depths may be provided in the proximal sections 21. By providing the notches 19 in this manner, the flexibility of the proximal sections 21 of the ridges 17 can be increased, and the rigidity of the ridges 17 can be ensured at the notches 19 that are relatively shallow. Therefore, the balloon 10 can be withdrawn and passed through a curved portion, while ensuring the scoring function provided by the ridges 17 in the proximal sections 21 of the ridges 17.

The deepest depth of the notches 19 provided in each of the proximal sections 21 may be 1.5 times or more, 1.8 times or more, or 2 times or more the deepest depth of the notch 19 provided in the corresponding intermediate section 22. As a result, the flexibility of the proximal sections 21 of the ridges 17 can be increased, and the followability at the proximal portions of the balloon 10 can be increased when the balloon 10 is withdrawn and passed through a curved portion. On the other hand, the deepest depth of the notches 19 provided in each of the proximal sections 21 may be 10 times or less, 8 times or less, or 5 times or less the deepest depth of the notch 19 provided in the intermediate section 22. This makes it possible to increase the flexibility of the intermediate sections 22 of the ridges 17.

The proximal sections 21 may be provided with the notches 19 as follows. That is, when each of the proximal section 21 is bisected in the longitudinal direction into a first proximal section 21A and a second proximal section 21B from the proximal side, it may be preferable that at least one of the notches 19 be provided in at least the second proximal section 21B. When multiple notches 19 are provided in the proximal section 21, it may be preferable that the notch 19 that has the deepest depth in the proximal section 21 is provided in the second proximal section 21B. By providing the notches 19 in this manner, even if the balloon 10 has a long length in the longitudinal direction, the overall flexibility of the balloon 10 can be more easily ensured. For example, the notch 19 that has the deepest depth in the proximal section 21 may be provided in the second proximal section 21B, and the notch 19 that has a shallower depth may be provided in the first proximal section 21A. This ensures the overall flexibility of the balloon 10, making it easier to withdraw and pass the balloon 10 through a curved portion while ensuring the scoring function provided the ridges 17 in the proximal sections 21 of the ridges 17.

Each of the ridges 17 may be provided with the notches 19 such that the number of the notches 19 provided in the distal section 23 is greater than the number of the notches 19 provided in the intermediate section 22. As a result, the flexibility of the distal portion of the balloon can be increased, making it further easier to advance and insert the balloon 10 into a curved portion. In addition, by providing a smaller number of the notches 19 in the intermediate section 22 of each of the ridges 17, the scoring function in the intermediate section 22 of the ridge 17 can be enhanced.

In the case where the notches 19 are also provided in the proximal sections 21 of the ridges 17, each of the ridges 17 may be provided with the notches 19 such that the number of the notches 19 provided in the distal section 23 and the number of the notches 19 provided in the proximal section 21 are each greater than the number of the notches 19 provided in the intermediate section 22. This makes it easier to insert the balloon 10 into a curved portion both when advancing the balloon 10 and when withdrawing the balloon 10 for insertion. In addition, the scoring function in the intermediate section 22 of each of the ridges 17 can be enhanced.

The notches 19 may be arranged on each of the ridges 17 at substantially equal intervals in the longitudinal direction. Even when the notches 19 are arranged in this manner, the deep notch 19 provided in the distal section 23 make it easier to advance and insert the balloon 10 into a curved portion. In addition, the deep notch 19 provided in the proximal section 21 makes it easier to withdraw and pass the balloon 10 through a curved portion.

The plurality of notches 19 are provided in each of the ridges 17, and the lengths of the plurality of notches 19 in the longitudinal direction may be the same or different from each other in the ridge 17. For example, the maximum length in the longitudinal direction of the notch 19 provided in the distal section 23 may be formed to be longer than the maximum length in the longitudinal direction of the notch 19 provided in the intermediate section 22. Alternatively, the length in the longitudinal direction of the deepest notch 19 provided in the distal section 23 may be formed to be longer than the length in the longitudinal direction of the deepest notch 19 provided in the intermediate section 22. When the notches 19 are formed in the ridges 17 in this manner, the balloon 10 can be bent more significantly in the distal sections 23 of the ridges 17. In addition, the scoring function provided by the ridges 17 can be more easily enhanced in the intermediate sections 22.

In the above case, in each of the ridges 17, the maximum length in the longitudinal direction of the notch 19 provided in the distal section 23 may be 1.5 times or more, 1.8 times or more, or 2 times or more the maximum length in the longitudinal direction of the notch 19 provided in the intermediate section 22. In addition, the maximum length in the longitudinal direction of the notch 19 provided in the distal section 23 may be 10 times or less, 8 times or less, or 5 times or less the maximum length in the longitudinal direction of the notch 19 provided in the intermediate section 22. Alternatively, the length in the longitudinal direction of the deepest notch 19 provided in the distal section 23 may be 1.5 times or more, 1.8 times or more, or 2 times or more the length in the longitudinal direction of the deepest notch 19 provided in the intermediate section 22. In addition, the length in the longitudinal direction of the deepest notch 19 provided in the distal section 23 may be 10 times or less, 8 times or less, or 5 times or less the length in the longitudinal direction of the deepest notch 19 provided in the intermediate section 22.

When the notches 19 are also provided in the proximal sections 21 of the ridges 17, the maximum length in the longitudinal direction of the notch 19 provided in each of the proximal sections 21 may be formed to be longer than the maximum length in the longitudinal direction of the notch 19 provided in the corresponding intermediate section 22. Alternatively, the length in the longitudinal direction of the deepest notch 19 provided in the proximal section 21 may be formed to be longer than the length in the longitudinal direction of the deepest notch 19 provided in the intermediate section 22. When the notches 19 are formed in the ridges 17 as described above, the balloon 10 can be bent more significantly in the proximal sections 21 of the ridges 17.

In the above case, the maximum length in the longitudinal direction of the notch 19 provided in the proximal section 21 may be 1.5 times or more, 1.8 times or more, or 2 times or more the maximum length in the longitudinal direction of the notch 19 provided in the intermediate section 22. In addition, the maximum length in the longitudinal direction of the notch 19 provided in the proximal section 21 may be 10 times or less, 8 times or less, or 5 times or less the maximum length in the longitudinal direction of the notch 19 provided in the intermediate section 22. Alternatively, the length in the longitudinal direction of the deepest notch 19 provided in the proximal section 21 may be 1.5 times or more, 1.8 times or more, or 2 times or more the length in the longitudinal direction of the deepest notch 19 provided in the intermediate section 22. In addition, the length in the longitudinal direction of the deepest notch 19 provided in the proximal section 21 may be 10 times or less, 8 times or less, or 5 times or less the length in the longitudinal direction of the deepest notch 19 provided in the intermediate section 22.

The length of the straight portion 13 of the balloon 10 in the longitudinal direction may be, for example, 4 mm or more, 10 mm or more, 20 mm or more, or 30 mm or more. However, as the length of the balloon 10 in the longitudinal direction increases, it becomes more difficult for the balloon 10 provided with the ridges 17 to pass through a curved portion. Therefore, from the viewpoint of more effectively achieving the effect of providing the notches 19 in the ridges 17, the length of the straight portion 13 of the balloon 10 in the longitudinal direction may be 50 mm or more, 60 mm or more, or 80 mm or more.

As shown in FIG. 12, each of the ridges 17 may be formed such that, in a cross section of the straight portion 13 perpendicular to the longitudinal direction, the width thereof decreases in a stepped manner from the base portion 17B toward the top portion 17A, and the notches 19 may be formed only in a portion of the ridge 17 that is formed in a stepped manner on the side where the top portion 17A is present. For example, each of the ridges 17 may include a first stepped portion 26 adjacent to the outer surface of the balloon main body 16 and a second stepped portion 27 closer to the top portion 17A than the first stepped portion 26, and the notch 19 may be formed in the second stepped portion 27 but not in the first stepped portion 26. Alternatively, the notch 19 that is deeper may be formed so as to extend across the second stepped portion 27 and the first stepped portion 26, and the notch 19 that is shallower may be formed in the second stepped portion 27 but not in the first stepped portion 26. The first stepped portion 26 and the second stepped portion 27 may be made of the same material or may be made of different materials from each other. For example, both the first stepped portion 26 and the second stepped portion 27 may be made of a resin, or the first stepped portion 26 may be made of a metal while the second stepped portion 27 may be made of a resin.

A drug may be held on the outer surface of the straight portion 13 of the balloon 10. The drug is not particularly limited as long as it is a pharmacologically active substance, and examples of such drugs include pharmaceutically acceptable drugs, such as gene therapy agents, non-gene therapy agents, small molecules, and cells. In particular, when a catheter is used for the purpose of suppressing restenosis of a blood vessel after treatment in angioplasty, an anti-restenosis agent, such as an antiproliferative agent or an immunosuppressive agent, may be used as the drug. Examples of such drugs include paclitaxel, sirolimus (rapamycin), everolimus, and zotarolimus.

This application claims the benefit of priority based on Japanese Patent Application No. 2023-065587, filed on Apr. 13, 2023. The entire disclosure of Japanese Patent Application No. 2023-065587, filed on Apr. 13, 2023 is incorporated herein by reference.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

### REFERENCE SIGNS LIST

1 balloon catheter
2 shaft
3 inner shaft
4 outer shaft, 4A proximal outer shaft, 4B distal outer shaft
5 hub
6 fluid injection portion
7 guidewire port
8 distal tip
9 radiopaque marker
10 balloon
11 proximal sleeve portion
12 proximal tapered portion
13 straight portion
14 distal tapered portion
15 distal sleeve portion
16 balloon main body
17 ridge, 17A top portion, 17B base portion
18 ridge segment
19 notch, 19A top portion, 19B bottom portion, 19P proximal end, 19D distal end
20 interrupted portion
21 proximal section, 21A first proximal section, 21B second proximal section
22 intermediate section
23 distal section, 23A first distal section, 23B second distal section
24 ridge-present region
25 ridge-absent region
26 first stepped portion
27 Second stepped portion

1. A balloon for a balloon catheter having a longitudinal direction extending from a proximal side to a distal side, and radial and circumferential directions, both orthogonal to the longitudinal direction, comprising:
a straight portion, a proximal tapered portion located further toward the proximal side than the straight portion, and a distal tapered portion located further toward the distal side than the straight portion, wherein:
the straight portion comprises a cylindrical balloon main body and a ridge provided on an outer surface of the balloon main body, the ridge protruding outward in the radial direction and extending in the longitudinal direction,
a plurality of notches arranged in the longitudinal direction are formed in the ridge,
when the ridge is equally divided in the longitudinal direction into three sections that are a proximal section, an intermediate section, and a distal section, at least one of the notches is provided in each of the distal section and the intermediate section, and
a deepest depth of the at least one of the notches in the distal section is deeper than a deepest depth of the at least one of the notches in the intermediate section.

2. The balloon according to claim 1, wherein:
the at least one of notches is provided in each of the proximal section, the intermediate section, and the distal section, and
the deepest depth of the at least one of the notches in the distal section and a deepest depth of the at least one of the notches in the proximal section are deeper than the deepest depth of the at least one of the notches in the intermediate section.

3. The balloon according to claim 1, wherein the number of the notches provided in the distal section is greater than the number of the notches provided in the intermediate section.

4. The balloon according to claim 2, wherein the number of the notches provided in the distal section and the number of the notches provided in the proximal section are each greater than the number of the notches provided in the intermediate section.

5. The balloon according to claim 1, wherein the plurality of the notches having different depths are provided in the distal section.

6. The balloon according to claim 2, wherein the plurality of the notches having different depths are provided in each of the proximal section and the distal section.

7. The balloon according to claim 1, wherein the ridge is made of a resin, a metal, or a combination of the resin and the metal.

8. The balloon according to claim 2, wherein the ridge is made of a resin, a metal, or a combination of the resin and the metal.

9. The balloon according to claim 1, wherein a length of the straight portion in the longitudinal direction is 50 mm or more.

10. The balloon according to claim 2, wherein a length of the straight portion in the longitudinal direction is 50 mm or more.

11. A balloon catheter comprising the balloon according to claim 1.

12. A balloon catheter comprising the balloon according to claim 2.
